(19) United States
(12) Patent Application Publication
(10) Pub. No.: US 2024/0371518 A1
(43) Pub. Date: Nov. 7, 2024

(54) Method and Apparatus for Predicting Relevance Degree, and Method and Apparatus for Training Machine Leaning Module

(71) Applicant: BOE TECHNOLOGY GROUP CO., LTD., Chaoyang District, Beijing (CN)

(72) Inventors: Sifan Wang, Beijing (CN); Shuobin Liang, Beijing (CN)

(21) Appl. No.: 18/247,590

(22) PCT Filed: May 27, 2022
(86) PCT No.: PCT/CN2022/095495

§ 371 Date: Apr. 3, 2023

(51) Int. Cl. G16H 50/20; G16H 70/40

(52) U.S. Cl. CPC G16H 50/20; G16H 70/40

(57) ABSTRACT

The present disclosure provides a method and an apparatus for predicting a relevance degree, and a method and an apparatus for training a machine learning model. The method for predicting a relevance degree includes constructing a heterogeneous matrix; processing the feature vector of the each medication and the feature vector of the each disease by using a first machine learning model to obtain a first predicted value of the relevance degree between the each medication and the each disease; processing the heterogeneous matrix by using a second machine learning model to obtain a relevance degree matrix; and obtaining a predicted result of the relevance degree between the each medication and the each disease according to the first predicted value of the relevance degree and the second predicted value of the relevance degree between the each medication and the each disease.

### CROSS-REFERENCE TO RELATED APPLICATIONS

This application is the United States National Phase of International Patent Application No. PCT/CN2022/095495, filed May 27, 2022, the disclosure of which is hereby incorporated by reference in its entirety.

### BACKGROUND OF THE INVENTION

### Field of the Invention

The present disclosure relates to the field of information technology, and in particular, to a method and an apparatus for predicting a relevance degree and a method and an apparatus for training a machine learning model.

### Description of Related Art

At present, in order to solve the problems of large investment cost, long consumption period, low marketing success rate and the like faced by the research and development of new medications, research and development personnel adopt a medication repositioning technology to mine the relevance relation between marketed medications and diseases, thereby being beneficial to discovering new indications of marketed medications.

In the prior art, the characteristics of the marketed medications and the diseases are analyzed by using a deep learning model to identify the relevance relation between the marketed medications and the diseases.

### SUMMARY OF THE INVENTION

According to a first aspect of the embodiments of the present disclosure, a method for predicting a relevance degree is provided, comprising: constructing a heterogeneous matrix, wherein the heterogeneous matrix comprises a first matrix representing the similarity of each two medications in a set of medications, a second matrix representing a similarity of each two diseases in a set of diseases, and a third matrix representing a relevance degree of each medication in the set of medications and each disease in the set of diseases; obtaining a feature vector of the each medication in the set of medications and a feature vector of the each disease in the set of diseases by using the heterogeneous matrix; processing the feature vector of the each medication and the feature vector of the each disease by using a first machine learning model to obtain a first predicted value of the relevance degree of the each medication and the each disease; processing the heterogeneous matrix by using a second machine learning model to obtain a relevance degree matrix, wherein the relevance degree matrix comprises a second predicted value of the relevance degree of the each medication and the each disease; and obtaining a predicted result of the relevance degree of the each medication and the each disease according to the first predicted value of the relevance degree and the second predicted value of the relevance degree of the each medication and the each disease.

In some embodiments, the prediction result of the relevance degree of an i^{th} medication and a j^{th} disease is a weighted sum of the first predicted value of the relevance degree of the i^{th} medication and the j^{th} disease and the second predicted value of the relevance degree of the i^{th} medication and the j^{th} disease, 1≤i≤M, 1≤j≤N, M is a total number of the medications, and N is a total number of the diseases.

In some embodiments, the feature vector of the i^{th} medication respectively comprises the similarity of the i^{th} medication and the each medication in the set of medications and the relevance degree of the i^{th} medication and the each disease in the set of diseases; and the feature vector of the j^{th} disease respectively comprises the relevance degree of the j^{th} disease and the each disease in the set of diseases, and the similarity of the j^{th} disease and the each medication in the set of medications.

In some embodiments, the processing the heterogeneous matrix comprises: generating a transformation matrix by using the heterogeneous matrix and an unit matrix; generating an embedded feature vector according to the transformation matrix, a degree matrix of the heterogeneous matrix and a feature matrix, wherein the feature matrix comprises matrix;
splitting the embedded feature vector into a medication embedded vector and a disease embedded vector; and generating the relevance degree matrix by using the medication embedded vector, a preset weight vector and the disease embedded vector.

In some embodiments, the embedded feature vector is: Y=\tilde{D}^{-1/2}\tilde{G}\tilde{D}^{-1/2}HW, wherein \tilde{G} is the transformation matrix, \tilde{D} is the degree matrix, H is the feature matrix, and W is a learnable weight.

In some embodiments, the generating the embedded feature vector comprises: generating a temporary feature vector according to the transformation matrix, the degree matrix of the heterogeneous matrix, the feature matrix and a first learnable weight; and generating the embedded feature vector according to the transformation matrix, the degree matrix of the heterogeneous matrix, the temporary feature vector and a second learnable weight.

In some embodiments, the temporary feature vector Y₀ is: Y₀=\tilde{D}^{-1/2}\tilde{G}\tilde{D}^{-1/2}H₀W₀ wherein \tilde{G} is the transformation matrix, \tilde{D} is the degree matrix, H₀ is the feature matrix, and W₀ is the first learnable weight; and the embedded feature vector Y₁ is: Y₁=\tilde{D}^{-1/2}\tilde{G}\tilde{D}^{-1/2}·σY₀·W₁, wherein σ is the predetermined parameter, W₁ is the second learnable weight.

In some embodiments, the feature matrix H₀ is

$${H}_{0} = ( \begin{matrix}0 & {M}_{DD} \\ {M}_{DD}^{T} & 0\end{matrix} ) ,$$

wherein M_{DD} is the third matrix.

In some embodiments, the processing the feature vector of the each medication and the feature vector of the each disease by using the first machine learning model comprises: splicing the feature vector of the each medication and the feature vector of the each disease to obtain a splicing feature; and processing the splicing feature by using a first machine learning model to obtain the first predicted value of the relevance degree of the each medication and the each disease.

In some embodiments, the constructing the heterogeneous matrix comprises: constructing a first matrix, wherein the first matrix comprises the similarity of the each two medications in the set of medications; constructing a second matrix, wherein the second matrix comprises the similarity of the each two diseases in the set of diseases; constructing a third matrix, wherein the third matrix comprises the relevance degree of the each medication in the set of medications and the each disease in the set of diseases; and generating the heterogeneous matrix by using the first matrix, the second matrix and the third matrix.

In some embodiments, the heterogeneous matrix G is

$$G = ( \begin{matrix}{M}_{Dr} & {M}_{DD} \\ {M}_{DD}^{T} & {M}_{Di}\end{matrix} ) ,$$

wherein M_{Dr} is the first matrix, M_{DD} is the second matrix, M_{Di} is the third matrix.

According to a second aspect of the embodiments of the present disclosure, there is provided a apparatus for predicting a relevance degree, comprising: a processor; and a memory coupled to the processor, storing program instructions which, when executed by the processor, cause the processor to implement the method of any of the embodiments described above.

According to a third aspect of the embodiments of the present disclosure, there is provided a method for training a machine learning model, comprising: constructing a heterogeneous matrix, wherein the heterogeneous matrix comprises a first matrix representing the similarity of each two medications in a set of medications, a second matrix representing a similarity of each two diseases in a set of diseases, and a third matrix representing a relevance degree of each medication in the set of medications and each disease in the set of diseases; obtaining a feature vector of the each medication in the set of medications and a feature vector of the each disease in the set of diseases by using the heterogeneous matrix; processing the feature vector of the each medication and the feature vector of the each disease by using a first machine learning model to obtain a first predicted value of the relevance degree of the each medication and the each disease; processing the heterogeneous matrix by using a second machine learning model to obtain a relevance degree matrix, wherein the relevance degree matrix comprises a second predicted value of the relevance degree of the each medication and the each disease; obtaining a predicted result of the relevance degree of the each medication and the each disease according to the first predicted value of the relevance degree and the second predicted value of the relevance degree of the each medication and the each disease; determining a loss function according to the prediction result of the relevance degree of the each medication and the each disease; and training the first machine learning model and the second machine learning model by using the loss function.

In some embodiments, the loss function is a weighted sum of prediction results of the relevance degree of the each medication in the set of medications and the each disease in the set of diseases.

In some embodiments, the loss function Loss is: Loss=−λ·Σ_{(i,j)∈Y}_{⁺}log Sᵢⱼ+Σ_{(i,j)∈Y}_{^{−}}log(1−Sᵢⱼ), wherein λ is a weight value, (i,j)∈Y⁺ indicates that an i^{th} medication and a j^{th} disease belong to an associated data Y⁺, (i,j)∈Y⁻ indicates that the i^{th} medication and the j^{th} disease belong to a non-associated data Y⁻, Sᵢⱼ is the prediction result of the relevance degree of the i^{th} medication and the j^{th} disease, 1≤i≤M, 1≤j≤N, M is the total number of the medications, and N is the total number of the diseases.

In some embodiments, the prediction result of the relevance degree of the i^{th} medication and the j^{th} disease is a weighted sum of the first predicted value of the relevance degree of the i^{t}h medication and the j^{th} disease and the second predicted value of the relevance degree of the i^{t}h medication and the j^{th} disease.

In some embodiments, the feature vector of the i^{th} medication respectively comprises the similarity of the i^{th} medication and the each medication in the set of medications and the relevance degree of the i^{th} medication and the each disease in the set of diseases; and the feature vector of the j^{th} disease respectively comprises the relevance degree of the j^{th} disease and the each disease in the set of diseases, and the similarity of the j^{th} disease and the each medication in the set of medications.

In some embodiments, the processing the heterogeneous matrix comprises: generating a transformation matrix by using the heterogeneous matrix and an unit matrix; generating an embedded feature vector according to the transformation matrix, a degree matrix of the heterogeneous matrix and a feature matrix, wherein the feature matrix comprises the third matrix; splitting the embedded feature vector into a medication embedded vector and a disease embedded vector; and generating the relevance degree matrix by using the medication embedded vector, a preset weight vector and the disease embedded vector.

In some embodiments, the embedded feature vector is: Y=\tilde{D}^{-1/2}\tilde{G}\tilde{D}^{-1/2}HW, wherein \tilde{G} is the transformation matrix, \tilde{D} is the degree matrix, H is the feature matrix, and W is a learnable weight.

In some embodiments, the generating the embedded feature vector comprises: generating a temporary feature vector according to the transformation matrix, the degree matrix of the heterogeneous matrix, the feature matrix and a first learnable weight; and generating the embedded feature vector according to the transformation matrix, the degree matrix of the heterogeneous matrix, the temporary feature vector and a second learnable weight.

In some embodiments, the temporary feature vector Y₀ is: Y₀=\tilde{D}^{-1/2}\tilde{G}\tilde{D}^{-1/2}H₀W₀, wherein \tilde{G} is the transformation matrix, \tilde{D} is the degree matrix, H₀ is the feature matrix, and W₀ is the first learnable weight; and the embedded feature vector Y₁ is: Y₁=\tilde{D}^{-1/2}\tilde{G}\tilde{D}^{-1/2}·σY₀·W₁, wherein σ is the predetermined parameter, W₁ is the second learnable weight.

In some embodiments, the feature matrix H₀ is:

$${H}_{0} = ( \begin{matrix}0 & {M}_{DD} \\ {M}_{DD}^{T} & 0\end{matrix} ) ,$$

wherein M_{DD} is the third matrix.

In some embodiments, the processing the feature vector of the each medication and the feature vector of the each disease by using the first machine learning model comprises: splicing the feature vector of the each medication and the feature vector of the each disease to obtain a splicing feature; and processing the splicing feature by using a first machine learning model to obtain the first predicted value of the relevance degree of the each medication and the each disease.

In some embodiments, the constructing the heterogeneous matrix comprises: constructing a first matrix, wherein the first matrix comprises the similarity of the each two medications in the set of medications; constructing a second matrix, wherein the second matrix comprises the similarity of the each two diseases in the set of diseases; constructing a third matrix, wherein the third matrix comprises the relevance degree of the each medication in the set of medications and the each disease in the set of diseases; and generating the heterogeneous matrix by using the first matrix, the second matrix and the third matrix.

In some embodiments, the heterogeneous matrix G is

$$G = ( \begin{matrix}{M}_{Dr} & {M}_{DD} \\ {M}_{DD}^{T} & {M}_{Di}\end{matrix} ) ,$$

wherein M_{Dr} is the first matrix, M_{DD} is the second matrix, M_{Di} is the third matrix.

According to a fourth aspect of embodiments of the present disclosure, there is provided a apparatus for training a machine learning module comprising: a memory configured to store instructions; a processor coupled to the memory, the processor configured to perform a training method implementing any of the embodiments described above based on instructions stored by the memory.

According to a fifth aspect of embodiments of the present disclosure, there is provided a non-transitory computer-readable storage medium, wherein the non-transitory computer-readable storage medium stores computer instructions, which when executed by a processor, implement the method according to any one of the embodiments described above.

Other features of the present disclosure and advantages thereof will become apparent from the following detailed description of exemplary embodiments thereof, which proceeds with reference to the accompanying drawings.

### BRIEF DESCRIPTION OF THE DRAWINGS

The accompanying drawings, which are incorporated in and constitute a part of this specification, illustrate embodiments of the disclosure and together with the description, serve to explain the principles of the disclosure.

The present disclosure may be more clearly understood from the following detailed description taken in conjunction with the accompanying drawings, in which:

FIG. 1 is a schematic flow chart diagram of a method for predicting a relevance degree according to one embodiment of the present disclosure;

FIG. 2 is a schematic flowchart of a method for constructing a heterogeneous matrix according to one embodiment of the present disclosure;

FIG. 3 is a schematic diagram of a heterogeneous matrix according to one embodiment of the present disclosure;

FIG. 4 is a schematic diagram of a heterogeneous matrix according to another embodiment of the present disclosure;

FIG. 5 is a schematic flowchart of a method for processing a heterogeneous matrix according to one embodiment of the present disclosure;

FIG. 6 is a schematic diagram of a relevance degree matrix according to one embodiment of the present disclosure;

FIG. 7 is a schematic structural diagram of a apparatus for predicting a relevance degree according to one embodiment of the present disclosure;

FIG. 8 is a schematic structural diagram of a method for training machine learning model according to one embodiment of the present disclosure;

FIG. 9 is a schematic structural diagram of a apparatus for training a machine learning model according to one embodiment of the present disclosure.

It should be understood that the dimensions of the various parts shown in the drawings are not drawn to scale. Further, the same or similar reference numerals denote the same or similar components.

### DESCRIPTION OF THE INVENTION

Various exemplary embodiments of the present disclosure will now be described in detail with reference to the accompanying drawings. The description of the exemplary embodiments is merely illustrative and is in no way intended to limit the disclosure, its application, or uses. The present disclosure may be embodied in many different forms and is not limited to the embodiments described herein. These embodiments are provided so that this disclosure will be thorough and complete, and will fully convey the scope of the disclosure to those skilled in the art. It should be noted that: the relative arrangement of parts and steps, the composition of materials and values set forth in these embodiments are to be construed as illustrative only and not as limiting unless otherwise specifically stated.

The use of “first”, “second” and similar words in this disclosure is not intended to indicate any order, quantity, or importance, but rather is used to distinguish one element from another. The word “comprising” or “comprises”, and the like, means that the element preceding the word comprises the element listed after the word, and does not exclude the possibility that other elements may also be included.

All terms (including technical or scientific terms) used herein have the same meaning as commonly understood by one of ordinary skill in the art to which this disclosure belongs unless specifically defined otherwise. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Techniques, methods, and apparatus known to one of ordinary skill in the relevant art may not be discussed in detail but are intended to be part of the specification where appropriate.

The inventors found out through research that, in the prior art, shallow features obtained by a random walk mode are generally adopted in the process of analyzing the features of the marketed medications and the diseases by using a deep learning model. Because the features used are relatively unitary, the relevance degree between the marketed medications and the diseases cannot be effectively mined.

Accordingly, the present disclosure provides a relevance degree prediction scheme capable of effectively mining the relevance degree between the marketed medications and the diseases by means of the explicit features and the implicit features.

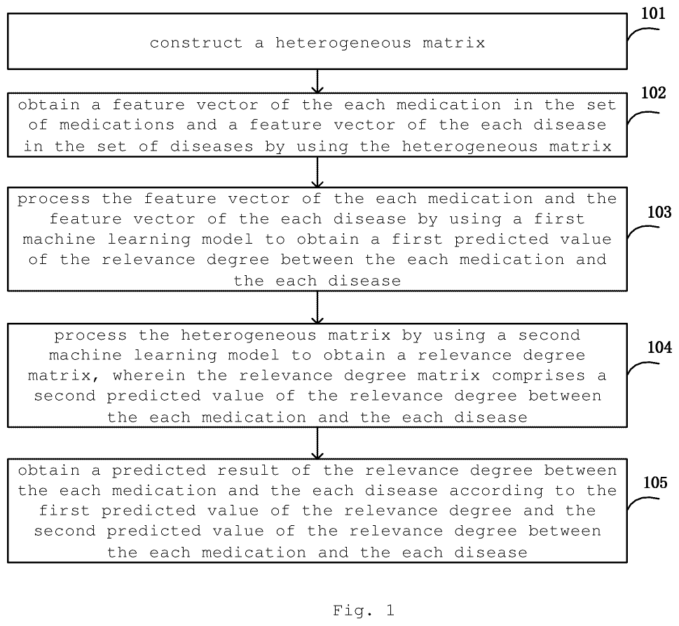

FIG. 1 is a schematic flowchart of a method for predicting a relevance degree according to one embodiment of the present disclosure. In some embodiments, the method for predicting a relevance degree is performed by a apparatus for predicting a relevance degree.

In step 101, a heterogeneous matrix is constructed, wherein the heterogeneous matrix comprises a first matrix representing the similarity between each two medications in a set of medications, a second matrix representing a similarity between each two diseases in a set of diseases, and a third matrix representing a relevance degree between each medication in the set of medications and each disease in the set of diseases.

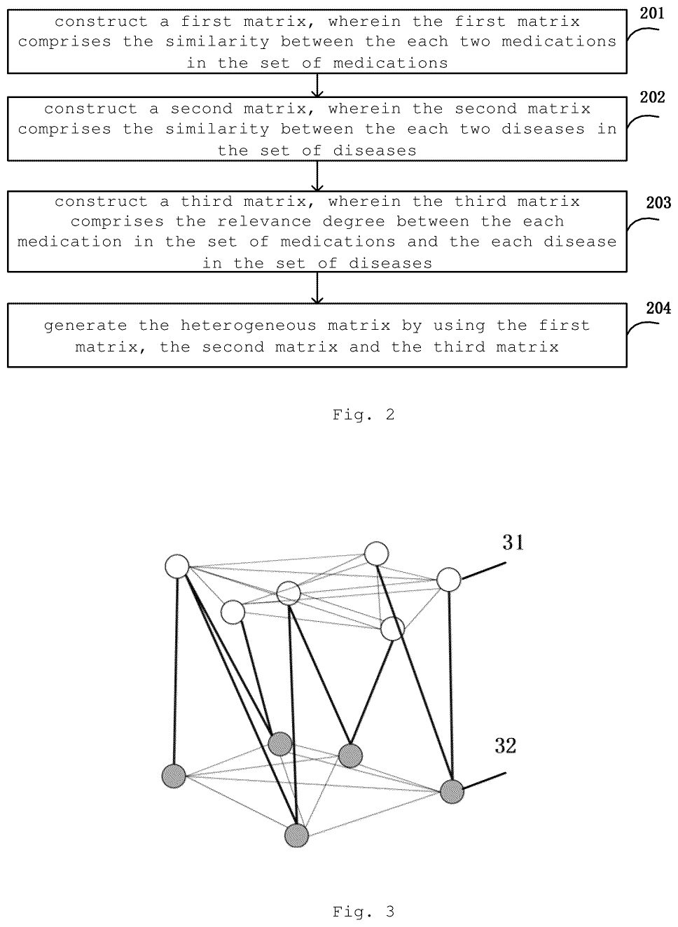

FIG. 2 is a schematic flowchart of a method for constructing a heterogeneous matrix according to one embodiment of the present disclosure. In some embodiments, the following method for constructing a heterogeneous matrix is performed by the apparatus for predicting a relevance degree.

At step 201, a first matrix is constructed, wherein the first matrix comprises the similarity between the each two medications in the set of medications.

It should be noted that the medication often has different properties, which are used to describe biological or chemical properties. The medication may be encoded as a binary feature vector, where each element implies the presence or absence of a feature descriptor. Because there are different types of features, it is possible to convert the medication into multiple types of feature vectors and calculate different medication-medication similarities from these features by using different similarity measures. For example, the relevance information between 881 chemical structures of the medication is collected by using the chemical structural features of the medications provided by a PubChem organic small molecule bioactivity database, wherein the structures adopt the Smiles standard and are shown in Table 1.

TABLE 1

| Feature | Type |
|---|---|
| vector | of |
| position | construction |
| 0 | >=4H |
| 1 | >=8H |
| . . . | . . . |
| 284 | C—C |
| . . . | . . . |
| 425 | P = O |
| . . . | . . . |
| 880 | BrC1C (Br) CCC1 |

The existence or non-existence of a certain chemical structure of the medication is represented by 1 and 0, and the formed one-dimensional vector is used as a feature vector of the drug, and the feature dimension is 881.

For example: the 2D chemical structure of the Smiles standard for the medication Acamprosate is represented as: CC(═O)NCCCS (═O)O. The corresponding 881 dimensional feature vector is:

11000000011000100011100000000000010000000000000000000000000000
00000000000000000000000000000000000000000000000000000000000000
00000000000000000000000000000000000000000000000000000000000000
00000000000000000000000000000000000000000000000000000000000000
00000000000000000000000000000000000111100000010000010000000010
00000000000000000000001000000000001100010111000000000001001000
00100000000000000010110000000000000010000010000010000000000000
00000100010000000100000111000001000000000000000000000000000000
00000000000000000000000000000000100000000000100000000000000000
00000000000000000000000000000000000000000000000000000001000000
00000000000000000000000101100000000010000000000000000000000000
00000000000000000000000000000000000000000000000000000000000000
00000000000000000000000000000000000000000000000000000000000000
00000000000000000000000000000000000000000000000000000000000000
0000000000000

In some embodiments, in the first matrix, the similarity of the each two medications in the set of medications comprises a Jaccard similarity or a cosine similarity of the two medications.

For example, in a case where the feature vector of medication i is xᵢ, and the feature vector of medication j is xⱼ, the Jaccard similarity Sᵢⱼ^{r} of the xᵢ and the xⱼ is shown in formula (1).

$${s}_{ij}^{r} = \frac{\vert {x}_{i} ⋂ {x}_{j} \vert}{\vert {x}_{i} ⋃ {x}_{j} \vert} \qquad ( 1 )$$

wherein |xᵢ∩xⱼ| denotes the number of times the xᵢ and the xⱼ are both 1 at the same positions, and |xᵢ∪xⱼ| denotes the number of times the xᵢ or the xⱼ is 1 at the same positions.

For another example, if the feature vector of the medication i is xᵢ, and the feature vector of the medication j is xⱼ, the cosine similarity Sᵢⱼ^{r} of the xᵢ and the xⱼ is as shown in formula (2).

$${S}_{ij}^{r} = \frac{{x}_{i} \cdot {x}_{j}}{‖ {x}_{i} ‖ ‖ {x}_{j} ‖} \qquad ( 2 )$$

wherein ∥xᵢ∥ denotes the L2 distance of the vector xᵢ, and ∥xⱼ∥ denotes the L2 distance of the vector xⱼ.

For example, in a case where there are M medications in the set of medications, Dr1 to DrM, the corresponding first matrix is shown in TABLE 2.

TABLE 2

| | Dr1 | Dr2 | . . . | DrM |
|---|---|---|---|---|
| Dr1 | 1 | 0.2 | . . . | 0.12 |
| Dr2 | 0.2 | 1 | . . . | 0.01 |
| . . . | . . . | . . . | . . . | . . . |
| DrM | 0.12 | 0.01 | . . . | 1 |

At step 202, a second matrix is constructed, wherein the second matrix comprises the similarity between the each two diseases in the set of diseases.

In some embodiments, in the second matrix, the

similarity of the each two diseases in the set of diseases comprises a semantic similarity of the each two diseases.

It should be noted that semantic similarity of the diseases is a measurement method for calculating the relationship between the diseases by using DAG (Directed Acyclic Graph). For example, a descriptive word list of diseases is searched out by using a MeSH (Medical Subject Headings) to establish a corresponding DAG. If most of the nodes of two diseases in the DAG are the same, it indicates that the two diseases have a higher semantic similarity.

For example, if there are N medications in the set of diseases, Di1 to DiM, the corresponding second matrix is shown in TABLE 3.

TABLE 3

| | Di1 | Di2 | . . . | DiN |
|---|---|---|---|---|
| Di1 | 1 | 0.7 | . . . | 0.23 |
| Di2 | 0.7 | 1 | . . . | 0.01 |
| . . . | . . . | . . . | . . . | . . . |
| DiN | 0.23 | 0.01 | . . . | 1 |

At step 203, a third matrix is constructed, wherein the third matrix comprises the relevance degree between the each medication in the set of medications and the each disease in the set of diseases.

For example, in a case where there is an relevance between a certain medication and a certain disease, the relevance degree is 1, otherwise it is 0. The corresponding third matrix is shown in TABLE 4.

TABLE 4

| | Di1 | Di2 | . . . | DiN |
|---|---|---|---|---|
| Dr1 | 0 | 0 | . . . | 0 |
| Dr2 | 0 | 1 | . . . | 0 |
| . . . | . . . | . . . | . . . | . . . |
| DrM | 1 | 0 | . . . | 1 |

At step 204, a heterogeneous matrix is generated by using the first matrix, the second matrix and the third matrix.

In some embodiments, the heterogeneous matrix G is

$$G = ( \begin{matrix}{M}_{Dr} & {M}_{DD} \\ {M}_{DD}^{T} & {M}_{Di}\end{matrix} ) \qquad ( 3 )$$

wherein M_{Dr} is the first matrix, M_{Di} is the second matrix, M_{DD} is the third matrix, and T represents transposition.

FIG. 3 is a schematic diagram of a heterogeneous matrix according to one embodiment of the present disclosure. As shown in FIG. 3, the heterogeneous matrix includes the similarity of the each two medications 31 in the set of medications, the similarity of the each two diseases 32 in the set of diseases, and the relevance degree of the each medication 31 and the each disease 32. Therefore, implicit features between medications and diseases can be obtained by means of the heterogeneous matrix.

Returning to FIG. 1, in step 102, a feature vector of the each medication in the set of medications and a feature vector of the each disease in the set of diseases are obtained by using the heterogeneous matrix.

For example, in the set of medications, the feature vector of the i^{th} medication respectively comprises the similarity of the i^{th} medication and the each medication in the set of medications and the relevance degree of the i^{th} medication and the each disease in the set of diseases, the feature vector of the j^{th} disease respectively comprises the relevance degree of the j^{th} disease and the each disease in the set of diseases, and the similarity of the j^{th} disease and the each medication in the set of medications, 1≤i≤M, 1≤j≤N, where M is the total number of the medications, and N is the total number of the diseases.

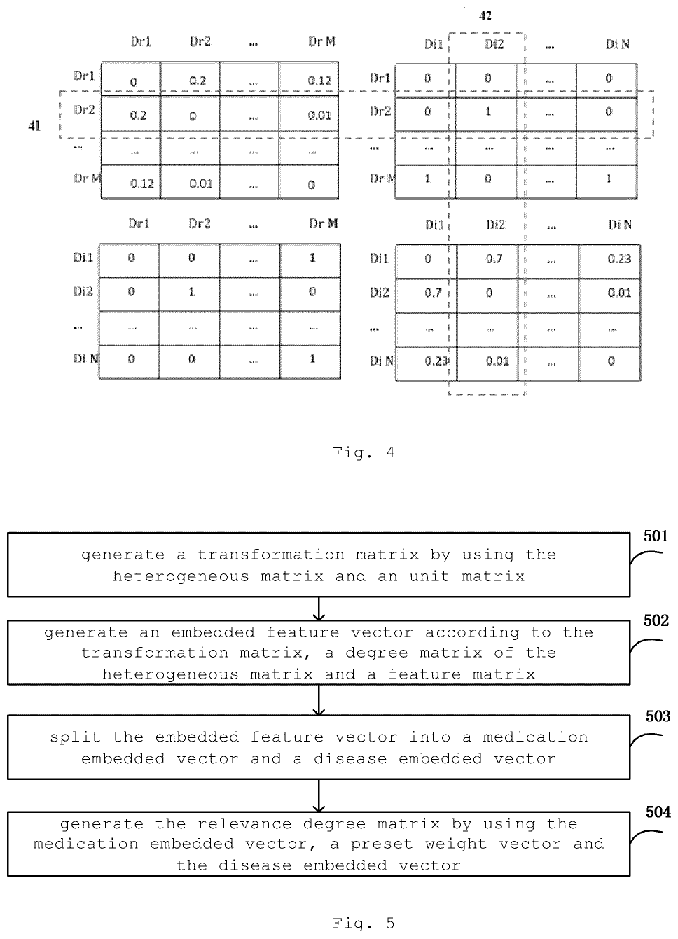

FIG. 4 is a schematic diagram of a heterogeneous matrix according to another embodiment of the present disclosure.

As shown in FIG. 4, the dashed box 41 includes the similarity between the medication Dr2 and the each medication in the set of medications, and the relevance degree of the medication Dr2 and the each disease in the set of diseases. The dashed box 42 includes the relevance degree of the disease Di2 and the each disease in the set of diseases, and the similarity between the disease Di2 and each medication in the set of medications, respectively.

Returning to FIG. 1, in step 103, the feature vector of the each medication and the feature vector of the each disease are processed by using a first machine learning model to obtain a first predicted value of the relevance degree between the each medication and the each disease.

For example, the first machine learning model is a LR (Logistic Regression) model.

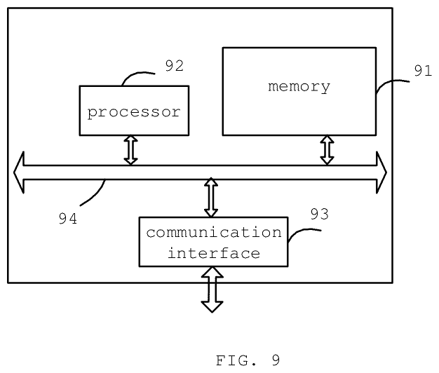

In some embodiments, the first machine learning model is trained bt using the embodiment shown in any of the embodiments of FIG. 8 below.

In some embodiments, the feature vector of the each medication and the feature vector of the each disease are spliced to obtain a splicing feature, and the splicing feature is processed by using a first machine learning model to obtain the first predicted value of the relevance degree of the each medication and the each disease.

For example, as shown in FIG. 4, the feature vector Fr2 of the medication Dr2 includes a similarity between the medication Dr2 and the each medication in the set of medications, and a relevance degree of the medication Dr2 and the each disease in the set of diseases. The feature vector Fi2 of the disease Di2 includes the relevance degree of the disease Di2 and the each disease in the set of diseases, and the similarity between the disease Di2 and the each medication in the set of medications. The feature vectors Fr2 and Fi2 are shown in TABLE 5.

TABLE 5

| | | |
|---|---|---|
| Fr2 | 0.2, 0, . . . , 0.01 | 0, 1, . . . , 0 |
| Fi2 | 0, 7, 0, . . . , 0.01 | 0, 1, . . . , 0 |

The Fr2 and the Fi2 are spliced to obtain the splicing features, and the splicing features are processed by using the first machine learning model to obtain the first predicted value of the relevance degree of the medication Dr2 and the disease Di2, namely, the explicit feature between the medication Dr2 and the disease Di2.

At step 104, the heterogeneous matrix is processed by a second machine learning model to obtain a relevance degree matrix, wherein the relevance degree matrix comprises a second predicted value of the relevance degree between the each medication and the each disease.

For example, the second machine learning model includes a GCNN (Graph Convolution Neural Network) model.

The GCNN can extract features from the graph data, and use the features to perform processing such as node classification, graph classification and link prediction on the graph data. The GCNN mainly includes a spectral-domain-based graph convolution method and a spatial-domain-based graph convolution method. The spectral-domain-based graph convolution method defines a graph convolution by introducing a filter from the perspective of graph signal processing, wherein the graph convolution operation is interpreted as removing noise from the graph signal. The spatial-domain-based graph convolution method represents the graph convolution as aggregating feature information from neighbors.

It should be noted here that the second machine learning model is essentially a dimension reduction representation of the features of the heterogeneous graph. The second machine learning model may therefore also include HetGNN (Heterogeneous Graph Neural Network), MetaPath2vec (meta path vector translation), RGCN (Relational Graph Convolutional Network), and so on.

In some embodiments, the second machine learning model is trained by adopting the embodiment shown in any of the embodiments of FIG. 8 below.

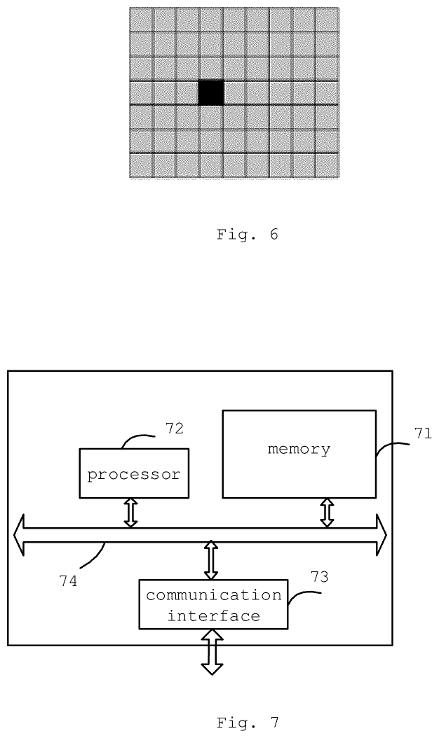

FIG. 5 is a schematic flowchart of a method for processing a heterogeneous matrix according to one embodiment of the present disclosure. In some embodiments, the following method for processing a heterogeneous matrix are performed by the apparatus for predicting a relevance degree.

In step 501, a transformation matrix is generated by using a heterogeneous matrix and a unit matrix.

For example, the transformation matrix \tilde{G} is shown in formula (4), wherein G is the heterogeneous matrix and I is the unit matrix.

$$\tilde{G} = G + I \qquad ( 4 )$$

In step 502, an embedded feature vector is generated according to the transformation matrix, the degree matrix of the heterogeneous matrix, and a feature matrix, wherein the feature matrix comprises a third matrix.

In some embodiments, the embedded feature vector Y is as shown in formula (5).

$$Y = {\tilde{D}}^{- 1 / 2} \tilde{G} {\tilde{D}}^{- 1 / 2} HW \qquad ( 5 )$$

wherein \tilde{G} is the transformation matrix, D is the degree matrix of the heterogeneous matrix, H is the feature matrix, W is a learnable weight. For example, the feature matrix H is shown in formula (6), and the learnable weight is shown in formula (7).

$${H}_{0} = ( \begin{matrix}0 & {M}_{DD} \\ {M}_{DD}^{T} & 0\end{matrix} ) \qquad ( 6 )$$

$$W ∈ {R}^{( N + M ) * k} \qquad ( 7 )$$

wherein N is the total number of the diseases, M is the total number of the medications, K is a preset parameter, and M_{DD} is a third matrix.

In other embodiments, a temporary feature vector is generated by using the transformation matrix, the degree matrix of the heterogeneous matrix, the feature matrix, and the first learnable weight, and the embedded feature vector is generated by using the transformation matrix, the degree matrix of the heterogeneous matrix, the temporary feature vector, and the second learnable weight.

For example, the temporary feature vector is as shown in equation (8).

$${Y}_{0} = {\tilde{D}}^{- 1 / 2} \tilde{G} {\tilde{D}}^{- 1 / 2} {H}_{0} {W}_{0} \qquad ( 8 )$$

wherein \tilde{G} is a transformation matrix, \tilde{D} is a degree matrix of the heterogeneous matrix, H₀ is the feature matrix, and W₀ is a first learnable weight. For example, the feature matrix H₀ is shown in formula (6).

The embedded feature vector Y₁ is shown in formula (9):

$${Y}_{1} = {\tilde{D}}^{- 1 / 2} \tilde{G} {\tilde{D}}^{- 1 / 2} \cdot σ {Y}_{0} \cdot {W}_{1} \qquad ( 9 )$$

wherein σ is the preset parameter, and W₁ is a second learnable weight.

At step 503, the embedded feature vector is split into a medication embedded vector and a disease embedded vector.

At step 504, the relevance degree matrix is generated by using the medication embedded vector, a preset weight vector and the disease embedded vector.

For example, the embedded feature vector Y is split into a medication embedded vector Y_{M} and a disease embedded vector Y_{N}, and the relevance degree matrix Y_{G} generated is shown in formula (10), wherein W′ is a preset weight vector.

$${Y}_{G} = {Y}_{M} \cdot {W}^{′} \cdot {Y}_{N} \qquad ( 10 )$$

FIG. 6 is a schematic diagram of a relevance degree matrix according to one embodiment of the present disclosure.

As shown in FIG. 6, the relevance degree matrix includes a second relevance degree predicted value between the each medication and the each disease. For example, the black block in FIG. 6 represents the relevance degree predicted value between the 4^{th} medication in the set of medications and the 4^{th} disease in the set of diseases.

Returning to FIG. 1, in step 105, a predicted result of the relevance degree between the each medication and the each disease is obtained according to the first predicted value of the relevance degree and the second predicted value of the relevance degree between the each medication and the each disease.

For example, the predicted result of the relevance degree of the i^{th} medication and the j^{th} disease is a weighted sum of the first predicted value of the relevance degree and the second predicted value of the relevance degree of the i^{th} medication and the j^{th} disease.

Set the first predicted value of the relevance degree of the i^{th} medication and the j^{th} disease as S_{LR} and set the second predicted value of the relevance degree of the i^{th} medication and the j^{th} disease as S_{GCN}, then the predicted result of the relevance degree of the i^{th} medication and the j^{th} disease is shown in a formula (11), wherein α is a weight value. For example, α is 0.45.

$${S}_{ij} = α {S}_{LR} + ( 1 - α ) {S}_{GCN} \qquad ( 11 )$$

In the method for predicting a relevance degree provided in the above embodiment of the present disclosure, by extracting the explicit features (i.e., surface features) between the medication and the disease, the association relation between the medication and the disease can be retained with optimal efficiency, and by extracting the implicit features between the medication and the disease, the deep topological features that cannot be extracted by using the surface features can be effectively extracted. By fusing the extracted explicit features and features, the advantages of both the explicit features and the implicit features can be taken into account, the association relation between the marketed medications and diseases is effectively excavated, and the accuracy of medication relocation is improved.

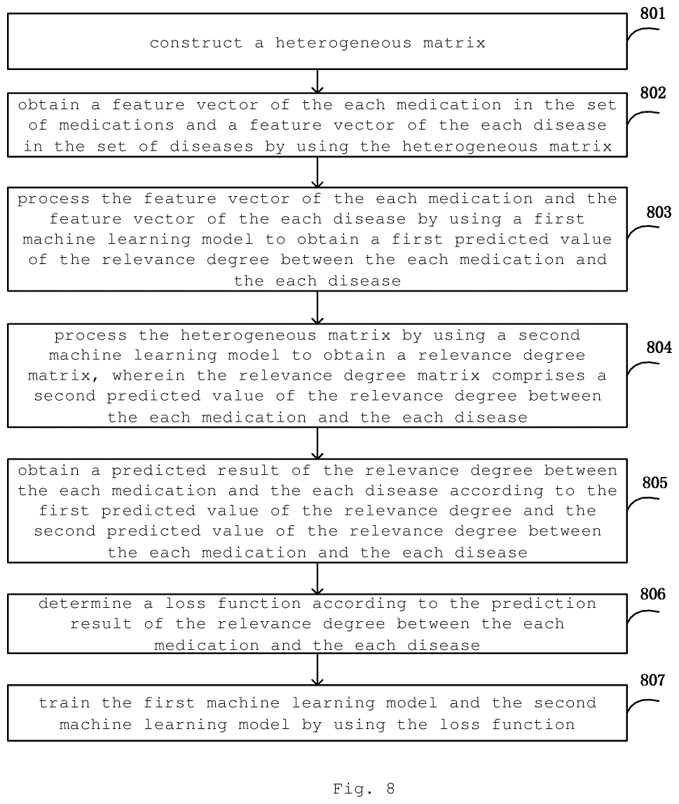

FIG. 7 is a schematic structural diagram of a apparatus for predicting a relevance degree according to one embodiment of the present disclosure. As shown in FIG. 7, the apparatus for predicting a relevance degree includes a memory 71 and a processor 72.

The memory 71 is used to store instructions. The processor 72 is coupled to a memory 71. The processor 72 is configured to perform a method as referred to in any of the embodiments of FIG. 1, 2 or 5 based on the instructions stored by the memory.

As shown in FIG. 7, the degree of association prediction apparatus further includes a communication interface 73 for information interaction with other devices. Meanwhile, the degree of association prediction apparatus further comprises a bus 74, and the processor 72, the communication interface 73 and the memory 71 are communicated with each other through the bus 74. The memory 71 may include a random access memory (RAM) and a non-volatile memory (NVM). Such as at least one disk storage. The memory 71 may also be a memory array. The storage 71 may also be partitioned and the blocks may be combined into virtual volumes according to certain rules.

Further, the processor 72 may be a central processing unit, or may be an ASIC (Application Specific Integrated Circuit), or one or more Integrated circuits configured to implement embodiments of the present disclosure.

The present disclosure also provides a non-transitory computer-readable storage medium. The computer readable storage medium stores computer instructions, and when executed by the processor, the instructions implement the method according to any one of FIG. 1, FIG. 2 or FIG. 5.

FIG. 8 is a schematic structural diagram of a method for training a machine learning model according to one embodiment of the present disclosure. In some embodiments, the following method for training a machine learning model is performed by a apparatus for training the machine learning model.

In step 801, a heterogeneous matrix is constructed, wherein the heterogeneous matrix comprises a first matrix representing the similarity between each two medications in a set of medications, a second matrix representing a similarity between each two diseases in a set of diseases, and a third matrix representing a relevance degree between each medication in the set of medications and each disease in the set of diseases.

For example, a heterogeneous matrix is constructed according to the embodiment shown in FIG. 2.

In step 802, the feature vector of each medication in the set of medications and the feature vector of each disease in the set of diseases are obtained by using the heterogeneous matrix.

For example, in the set of medications, the feature vector of the i^{th} medication respectively includes the similarity between the i^{th} medication and the each medication in the set of medications, and the relevance degree between the i^{th} medication and the each disease in the set of diseases. The feature vector of the j^{th} disease respectively comprises the relevance degree between the j^{th} disease and the each disease in the set of diseases, and the similarity between the j^{th} disease and the each medication in the set of medications.

In step 803, the feature vector of the each medication and the feature vector of the each disease are processed by using a first machine learning model to obtain a first predicted value between the relevance degree of the each medication and the each disease.

For example, the first machine learning model is the LR model.

In some embodiments, the feature vector of the each medication and the feature vector of the each disease are spliced to obtain spliced features, and the spliced features are then processed by using a first machine learning model to obtain the first predicted value of the relevance degree between the each medication and the each disease.

At step 804, the heterogeneous matrix is processed by a second machine learning model to obtain a relevance degree matrix, wherein the relevance degree matrix comprises a second predicted value of the relevance degree between the each medication and the each disease.

In some embodiments, the second machine learning model comprises a graph convolution neural network model.

For example, the heterogeneous matrix is processed according to the embodiment shown in FIG. 5.

In step 805, the predicted result of the relevance degree between the each medication and the each disease is obtained according to the first predicted value of the relevance degree and the second predicted value of the relevance degree between the each medication and the each disease.

For example, the predicted result of the relevance degree of the i^{th} medication and the j^{th} disease is a weighted sum of the first predicted value of the relevance degree and the second predicted value of the relevance degree of the i^{th} medication and the j^{th} disease.

In some embodiments, the predicted result of the relevance degree between the each medication and the each disease is calculated by using the formula (11).

At step 806, a loss function is determined according to the prediction result of the relevance degree between the each medication and the each disease.

In some embodiments, the loss function is the weighted sum of the prediction result of the relevance degree between the each medication and the each disease.

For example, the loss function Loss is shown in formula (12).

$$Loss = - λ \cdot {\sum }_{( i , j ) ∈ {Y}^{+}} log {S}_{ij} + {\sum }_{( i , j ) ∈ {Y}^{-}} log ( 1 - {S}_{ij} ) \qquad ( 12 )$$

wherein λ is a weight value, (i,j)∈Y⁺ indicates that an i^{th} medication and a j^{th} disease belong to an associated data Y⁺, (i,j)∈Y⁻ indicates that the i^{th} medication and the j^{th} disease belong to a non-associated data Y⁻, Sᵢⱼ is the prediction result of the relevance degree of the i^{th} medication and the j^{th} disease, 1≤i≤M, 1≤j≤N, M is the total number of the medications, and N is the total number of the diseases.

In step 807, the first machine learning model and the second machine learning model are trained by using a loss function.

FIG. 9 is a schematic structural diagram of a apparatus for training a machine learning model according to one embodiment of the present disclosure. As shown in FIG. 9, the apparatus for training a machine learning model includes a memory 91, a processor 92, a communication interface 93, and a bus 74. FIG. 9 differs from FIG. 7 in that, in the embodiment shown in FIG. 9, the processor 92 performs a method implementing any of the embodiments of FIG. 8 based on instructions stored by the memory 91.

So far, embodiments of the present disclosure have been described in detail. Some details well known in the art have not been described in order to avoid obscuring the concepts of the present disclosure. Those skilled in the art can now fully appreciate how to implement the teachings disclosed herein, in view of the foregoing description.

Although some specific embodiments of the present disclosure have been described in detail by way of example, it should be understood by those skilled in the art that the above examples are for illustration only and are not intended to limit the scope of the present disclosure. It will be understood by those skilled in the art that various changes may be made and equivalents may be substituted for elements thereof without departing from the scope and spirit of the present disclosure. The scope of the present disclosure is defined by the appended claims.

1. A method for predicting a relevance degree, comprising:
constructing a heterogeneous matrix, wherein the heterogeneous matrix comprises a first matrix representing the similarity between each two medications in a set of medications, a second matrix representing a similarity between each two diseases in a set of diseases, and a third matrix representing a relevance degree between each medication in the set of medications and each disease in the set of diseases;
obtaining a feature vector of the each medication in the set of medications and a feature vector of the each disease in the set of diseases by using the heterogeneous matrix;
processing the feature vector of the each medication and the feature vector of the each disease by using a first machine learning model to obtain a first predicted value of the relevance degree between the each medication and the each disease;
processing the heterogeneous matrix by using a second machine learning model to obtain a relevance degree matrix, wherein the relevance degree matrix comprises a second predicted value of the relevance degree between the each medication and the each disease; and
obtaining a predicted result of the relevance degree between the each medication and the each disease according to the first predicted value of the relevance degree and the second predicted value of the relevance degree between the each medication and the each disease.

2. The method according to claim 1, wherein
the prediction result of the relevance degree between an i^{th} medication and a j^{th} disease is a weighted sum of the first predicted value of the relevance degree between the i^{th} medication and the j^{th} disease and the second predicted value of the relevance degree of the i^{th} medication and the j^{th} disease, 1≤i≤M, 1≤j≤N, M is a total number of the medications, and N is a total number of the diseases.

3. The method according to claim 2, wherein:
the feature vector of the i^{th} medication respectively comprises the similarity between the i^{th} medication and the each medication in the set of medications and the relevance degree between the i^{th} medication and the each disease in the set of diseases; and
the feature vector of the j^{th} disease respectively comprises the relevance degree between the j^{th} disease and the each disease in the set of diseases, and the similarity between the j^{th} disease and the each medication in the set of medications.

4. The method according to claim 1, wherein the processing the heterogeneous matrix comprises:
generating a transformation matrix by using the heterogeneous matrix and an unit matrix;
generating an embedded feature vector according to the transformation matrix, a degree matrix of the heterogeneous matrix and a feature matrix, wherein the feature matrix comprises the third matrix;
splitting the embedded feature vector into a medication embedded vector and a disease embedded vector; and
generating the relevance degree matrix by using the medication embedded vector, a preset weight vector and the disease embedded vector.

5. (canceled)

6. The method according to claim 4, wherein the generating the embedded feature vector comprises:
generating a temporary feature vector according to the transformation matrix, the degree matrix of the heterogeneous matrix, the feature matrix and a first learnable weight; and
generating the embedded feature vector according to the transformation matrix, the degree matrix of the heterogeneous matrix, the temporary feature vector and a second learnable weight.

7. (canceled)

8. (canceled)

9. The method according to claim 1, wherein the processing the feature vector of the each medication and the feature vector of the each disease by using the first machine learning model comprises:
splicing the feature vector of the each medication and the feature vector of the each disease to obtain a splicing feature; and
processing the splicing feature by using a first machine learning model to obtain the first predicted value of the relevance degree of the each medication and the each disease.

10. The method according to claim 1, wherein the constructing the heterogeneous matrix comprises:
constructing a first matrix, wherein the first matrix comprises the similarity between the each two medications in the set of medications;
constructing a second matrix, wherein the second matrix comprises the similarity between the each two diseases in the set of diseases;
constructing a third matrix, wherein the third matrix comprises the relevance degree between the each medication in the set of medications and the each disease in the set of diseases; and
generating the heterogeneous matrix by using the first matrix, the second matrix and the third matrix.

11. (canceled)

12. A apparatus for predicting a relevance degree, comprising:
a processor; and
a memory coupled to the processor, storing program instructions which, when executed by the processor, cause the processor to
construct a heterogeneous matrix, wherein the heterogeneous matrix comprises a first matrix representing the similarity between each two medications in a set of medications, a second matrix representing a similarity between each two diseases in a set of diseases, and a third matrix representing a relevance degree between each medication in the set of medications and each disease in the set of diseases;
obtain a feature vector of the each medication in the set of medications and a feature vector of the each disease in the set of diseases by using the heterogeneous matrix;
process the feature vector of the each medication and the feature vector of the each disease by using a first machine learning model to obtain a first predicted value of the relevance degree between the each medication and the each disease;
process the heterogeneous matrix by using a second machine learning model to obtain a relevance degree matrix, wherein the relevance degree matrix comprises a second predicted value of the relevance degree between the each medication and the each disease; and
obtain a predicted result of the relevance degree between the each medication and the each disease according to the first predicted value of the relevance degree and the second predicted value of the relevance degree between the each medication and the each disease.

13. (canceled)

14. The apparatus according to claim 26, wherein
the loss function is a weighted sum of prediction results of the relevance degree of the each medication in the set of medications and the each disease in the set of diseases.

15. (canceled)

16. The apparatus according to claim 14, wherein
the prediction result of the relevance degree of an i^{th} medication and a j^{th} disease is a weighted sum of the first predicted value of the relevance degree between the i^{t}h medication and the j^{th} disease and the second predicted value of the relevance degree between the i^{t}h medication and the j^{th} disease, 1≤i≤M, 1≤j≤N, M is a total number of the medications, and N is a total number of the diseases.

17. The apparatus according to claim 14, wherein:
the feature vector of the i^{th} medication respectively comprises the similarity between the i^{th} medication and the each medication in the set of medications and the relevance degree between the i^{th} medication and the each disease in the set of diseases; and
the feature vector of the j^{th} disease respectively comprises the relevance degree between the j^{th} disease and the each disease in the set of diseases, and the similarity between the j^{th} disease and the each medication in the set of medications.

18. The apparatus according to claim 26, wherein the processor is configured to:
generate a transformation matrix by using the heterogeneous matrix and an unit matrix;
generate an embedded feature vector according to the transformation matrix, a degree matrix of the heterogeneous matrix and a feature matrix, wherein the feature matrix comprises the third matrix;
split the embedded feature vector into a medication embedded vector and a disease embedded vector; and
generate the relevance degree matrix by using the medication embedded vector, a preset weight vector and the disease embedded vector.

19. (canceled)

20. The apparatus according to claim 18, wherein the processor is configured to:
generate a temporary feature vector according to the transformation matrix, the degree matrix of the heterogeneous matrix, the feature matrix and a first learnable weight; and
generate the embedded feature vector according to the transformation matrix, the degree matrix of the heterogeneous matrix, the temporary feature vector and a second learnable weight.

21. (canceled)

22. (canceled)

23. The apparatus according to claim 26, wherein the processor is configured to:
splice the feature vector of the each medication and the feature vector of the each disease to obtain a splicing feature; and
process the splicing feature by using a first machine learning model to obtain the first predicted value of the relevance degree of the each medication and the each disease.

24. The apparatus according to claim 26, wherein the processor is configured to:
construct a first matrix, wherein the first matrix comprises the similarity of the each two medications in the set of medications;
construct a second matrix, wherein the second matrix comprises the similarity of the each two diseases in the set of diseases;
construct a third matrix, wherein the third matrix comprises the relevance degree between the each medication in the set of medications and the each disease in the set of diseases; and
generate the heterogeneous matrix by using the first matrix, the second matrix and the third matrix.

25. (canceled)

26. A apparatus for training a machine learning model, comprising:
a processor; and
a memory coupled to the processor, storing program instructions which, when executed by the processor, cause the processor to:
construct a heterogeneous matrix, wherein the heterogeneous matrix comprises a first matrix representing the similarity between each two medications in a set of medications, a second matrix representing a similarity between each two diseases in a set of diseases, and a third matrix representing a relevance degree between each medication in the set of medications and each disease in the set of diseases;
obtain a feature vector of the each medication in the set of medications and a feature vector of the each disease in the set of diseases by using the heterogeneous matrix;
process the feature vector of the each medication and the feature vector of the each disease by using a first machine learning model to obtain a first predicted value of the relevance degree between the each medication and the each disease;
process the heterogeneous matrix by using a second machine learning model to obtain a relevance degree matrix, wherein the relevance degree matrix comprises a second predicted value of the relevance degree between the each medication and the each disease; and
obtain a predicted result of the relevance degree between the each medication and the each disease according to the first predicted value of the relevance degree and the second predicted value of the relevance degree between the each medication and the each disease;
determine a loss function according to the prediction result of the relevance degree between the each medication and the each disease; and
train the first machine learning model and the second machine learning model by using the loss function.

27. A non-transitory computer-readable storage medium, wherein the computer-readable storage medium stores computer instructions that, when executed by a processor, implement the method of claim 1.
